(19) United States
(12) Patent Application Publication
(10) Pub. No.: US 2003/0096880 A1
(43) Pub. Date: May 22, 2003

(54) Combustion deposited metal-metal oxide catalysts and process for producing synthesis gas

(75) Inventors: Daxiang Wang, Ponca City, OK (US); Yaming Jin, Ponca City, OK (US); Harold A. Wright, Ponca City, OK (US); Rafael L. Espinoza, Ponca City, OK (US)

Correspondence Address: DAVID W. WESTPHAL, CONOCOPHILLIPS COMPNAY, P.O. BOX 1267, PONCA CITY, OK, 74602-1267, US

(73) Assignee: Conoco Inc., Houston, TX

(21) Appl. No.: 10/282,382

(22) Filed: Oct. 29, 2002

Related U.S. Application Data
(60) Provisional application No. 60/336,472, filed on Nov. 2, 2001.

(51) Int. Cl. B01J 31/00; C07C 27/06; B01J 23/00

(52) U.S. Cl. 518/714; 502/162; 502/325; 502/304; 423/651; 518/715

(57) ABSTRACT

Combustion dispersed metal-metal oxide catalysts that are highly active for catalyzing the net partial oxidation of methane to CO and H₂ are disclosed, along with their manner of making and processes for producing synthesis gas employing the new catalysts. A preferred catalyst comprises rhodium nanoparticles, with or without a rare earth promoter, that is deposited on α-alumina by combusting a mixture of catalyst precursor materials and a flammable organic compound. In a preferred syngas production process a stream of reactant gas mixture containing methane and O₂ is passed over the catalyst in a short contact time reactor to efficiently produce a mixture of carbon monoxide and hydrogen at superatmospheric pressures.

### CROSS REFERENCE TO RELATED APPLICATIONS

This application claims the benefit under 35 U.S.C. §119(e) of U.S. Provisional Patent Application No. 60/336,472 filed Nov. 2, 2001, the disclosure of which is hereby incorporated herein by reference.

### BACKGROUND OF THE INVENTION

1. Field of the Invention

The present invention generally relates to processes for the conversion of light hydrocarbons (e.g., methane and natural gas) to a mixture of carbon monoxide and hydrogen (“synthesis gas” or “syngas”) using metal-metal oxide catalysts. More specifically, the invention relates to the preparation of metal oxide supported highly dispersed metal or mixed oxide catalysts prepared by combusting a mixture of catalytic and support precursor compounds, and to the use of the combustion synthesized catalysts for generating synthesis gas.

2. Description of Related Art

Large quantities of methane, the main component of natural gas, are available in many areas of the world, and natural gas is predicted to outlast oil reserves by a significant margin. However, most natural gas is situated in areas that are geographically remote from population and industrial centers. The costs of compression, transportation, and storage make its use economically unattractive.

To improve the economics of natural gas use, much research has focused on methane as a starting material for the production of higher hydrocarbons and hydrocarbon liquids. The conversion of methane to hydrocarbons is typically carried out in two steps. In the first step, methane is reformed with water to produce carbon monoxide and hydrogen (i.e., synthesis gas or syngas). In a second step, the syngas intermediate is converted to higher hydrocarbon products by processes such as the Fischer-Tropsch Synthesis. For example, fuels with boiling points in the middle distillate range, such as kerosene and diesel fuel, and hydrocarbon waxes may be produced from the synthesis gas.

Current industrial use of methane as a chemical feedstock proceeds by the initial conversion of methane to carbon monoxide and hydrogen by either steam reforming or dry reforming. Steam reforming currently is the major process used commercially for the conversion of methane to synthesis gas, the reaction proceeding according to Equation 1.

CH₄+H₂O⇄CO+3H₂ (1)

Although steam reforming has been practiced for over five decades, efforts to improve the energy efficiency and reduce the capital investment required for this technology continue. The steam reforming reaction is endothermic (about 49 kcal/mol), requiring the expenditure of large amounts of fuel to produce the necessary heat for the industrial scale process. Another drawback of steam reforming is that for many industrial applications, the 3:1 ratio of H₂:CO products is problematic, and the typically large steam reforming plants are not practical to set up at remote sites of natural gas formations.

The catalytic partial oxidation (“CPOX”) of hydrocarbons, e.g., methane or natural gas, to syngas has also been described in the literature. In catalytic partial oxidation, natural gas is mixed with air, oxygen-enriched air, or oxygen, and introduced to a catalyst at elevated temperature and pressure. The partial or direct oxidation of methane yields a syngas mixture with a H₂: CO ratio of 2:1, as shown in Equation 2.

CH₄+½O₂→CO+2H₂ (2)

This ratio is more useful than the H₂:CO ratio from steam reforming for the downstream conversion of the syngas to chemicals such as methanol or to fuels. The CPOX reaction is exothermic (−8.5 kcal/mol), in contrast to the strongly endothermic steam reforming reaction. Furthermore, oxidation reactions are typically much faster than reforming reactions. This allows the use of much smaller reactors for catalytic partial oxidation processes than is possible in a conventional steam reforming process.

While its use is currently limited as an industrial process, CPOX of methane has recently attracted much attention due to its inherent advantages, such as the fact that due to the significant heat that is released during the process, there is no requirement for the continuous input of heat in order to maintain the reaction, in contrast to steam reforming processes. An attempt to overcome some of the disadvantages and costs typical of steam reforming by production of synthesis gas via the catalytic partial oxidation of methane is described in European Patent No. 303,438. According to that method, certain high surface area monoliths coated with metals or metal oxides that are active as oxidation catalysts, e.g., Pd, Pt, Rh, Ir, Os, Ru, Ni, Cr, Co, Ce, La, and mixtures thereof, are employed as catalysts. Other suggested coating metals are noble metals and metals of groups IA, IIA, III, IV, VB, VIB, or VIIB of the periodic table of the elements.

Other methane oxidation reactions include the highly exothermic combustion (−192 kcal/mol) and partial combustion (−124 kcal/mol) reactions, Equations 3 and 4, respectively.

CH₄+2O₂→CO₂+2H₂O (3)

CH₄+3/2O₂→CO+2H₂O (4)

U.S. Pat. No. 5,149,464 describes a method for selectively converting methane to syngas at 650-950° C. by contacting a methane/oxygen mixture with a solid catalyst which is a d-block transition metal on a refractory support, an oxide of a d-block transition metal, or a compound of the formula MₓM′_{y}O_{z} wherein M′ is a d-block transition metal and M is Mg, B, Al, Ga, Si, Ti, Zr, Hf or a lanthanide.

U.S. Pat. No. 5,500,149 describes the combination of dry reforming and partial oxidation of methane, in the presence of added CO₂ to enhance the selectivity and degree of conversion to synthesis gas. The catalyst is a d-block transition metal or oxide such as a group VIII metal on a metal oxide support such as alumina, is made by precipitating the metal oxides, or precursors thereof such as carbonates or nitrates or any thermally decomposable salts, onto a refractory solid which may itself be massive or particulate; or one metal oxide or precursor may be precipitated onto the other. Preferred catalyst precursors are those having the catalytic metal highly dispersed-on an inert metal oxide support and in a form readily reducible to the elemental state.

For successful commercial scale operation a catalytic partial oxidation process must be able to achieve a high conversion of the methane feedstock at high gas hourly space velocities, and the selectivity of the process to the desired products of carbon monoxide and hydrogen must be high. Although there have been advances toward the development of commercially feasible CPOX processes, most of the known systems operate at high reaction temperatures, usually above 500° C. and even as high as 1,300° C. and are highly exothermic. Because of the high reaction temperatures, especially at the catalyst surface, the catalyst must possess thermal stability.

In most of the existing syngas production processes it is difficult to select a catalyst that will be economical for large scale industrial use, yet will provide the desired level of activity and selectivity for CO and H₂ and demonstrate long on-stream life. Moreover, such high conversion and selectivity levels must be achieved without detrimental effects to the catalyst, such as the formation of carbon deposits (“coke”) on the catalyst, which severely reduces catalyst performance. Also, the choice of catalyst composition and the manner in which the catalyst is made are important factors in determining whether a catalyst will have sufficient physical and chemical stability to operate satisfactorily for extended periods of time on stream at moderate to high temperatures and will avoid high pressure drop in a syngas production operation.

Today, metal oxide supported noble metal catalysts or mixed metal oxide catalysts are most commonly used for the selective oxidation of hydrocarbons and for catalytic combustion processes. Various techniques are employed to prepare the catalysts, including impregnation, washcoating, xerogel, aerogel or sol gel formation, spray drying and spray roasting. In addition to catalyst powders and pellets, extrudates and monolith supports having pores or longitudinal channels or passageways are commonly used. Such catalyst forming techniques and configurations are well described in the literature, for example, in Structured Catalysts and Reactors, A. Cybulski and J. A. Moulijn (Eds.), Marcel Dekker, Inc., 1998, p. 599-615 (Ch. 21, X. Xu and J. A. Moulijn, “Transformation of a Structured Carrier into Structured Catalyst”).

U.S. Pat. No. 5,510,056 discloses a ceramic foam supported Ru, Rh, Pd, Os, Ir or Pt catalyst having a specified tortuosity and number of interstitial pores that is said to allow operation at high gas space velocity. The catalyst is prepared by depositing the metal on a carrier using an impregnation technique, which typically comprises contacting the carrier material with a solution of a compound of the catalytically active metal, followed by drying and calcining the resulting material. The catalyst is employed for the catalytic partial oxidation of a hydrocarbon feedstock.

U.S. Pat. No. 5,648,582 discloses a rhodium or platinum catalyst prepared by washcoating an alumina foam monolith having an open, cellular, sponge-like structure. The catalyst is used for the catalytic partial oxidation of methane at space velocities of 120,000 hr.⁻¹ to 12,000,000 hr.⁻¹

Vernon, D. F. et al. (Catalysis Letters 6:181-186 (1990)) describe the partial oxidation of methane to synthesis gas using various transition metal catalysts such as Pd, Pt, Ru or Ni on alumina, or certain transition metal oxides including Pr₂Ru₂O₇ and Eu₂Ir₂O₇, under a range of conditions. The supported catalysts are prepared by the incipient wetness technique. The appropriate amount of metal chloride, dissolved in a minimal amount of dilute HCl, is added in aliquots to alumina mesh, removing the solvent under reduced pressure after each addition.

U.S. Pat. No. 5,447,705 discloses a mixed metal oxide catalyst for the partial oxidation of methane. The catalyst has a perovskite crystalline structure and the general composition: LnₓA_{1-y}B_{y}O₃ wherein Ln is a lanthanide and A and B are different metals chosen from Group UVb, Vb, VIb, VIIb or VIII of the Periodic Table of the Elements. The catalyst is prepared from an aqueous solution S containing soluble compounds of Ln and/or A and/or B in proportions corresponding to those of a desired formulation. A solution containing a complexing acid is added to produce a precipitate containing Ln, A and B, after which the residual solvent is separated and the precipitate is dried and calcined at a temperature of between 200° C. and 900° C.

In the pursuit of ultra-fine, high surface area catalysts for treatment of automobile exhaust, a combustion method for preparing α-alumina supported Pt, Pd, Ag and Au metal particles was devised (Bera et al. J. Mater. Chem. (1999) 9:1801-1805). In that study, combustion of aqueous redox mixtures containing metal salts and urea yield nearly spherical metal particles of uniform size (i.e., 7, 12, 20 and 15 nm, respectively) dispersed on alumina. The catalysts are active for catalyzing the complete oxidation of CO and NO. The same catalysts are also shown to be active for catalyzing the complete oxidation of CH₄ and C₃H₆ to CO₂ (P. Bera et al. Phys. Chem. Chem. Phys. (2000) 2:373-378). The homogeneously dispersed nanosize metal particles prepared by a single step combustion method provide the active sites for catalysis.

The synthesis of certain CeO₂ supported Pt and Pd catalysts using an aqueous solution of catalyst precursors and oxalyldihydrazide has also been described (P. Bera et al. J. Catalysis (2000) 196:293-301). The solution is heated in an open vessel until dehydrated and surface ignition of the residue occurs. The resulting product, an ionic dispersion of Pt or Pd on CeO₂, was active for catalyzing nitrous oxide reduction, oxidation of carbon monoxide, and the complete combustion of hydrocarbons, all of which is applicable to cleaning up automobile exhaust. A similar combustion technique has also been used to prepare certain Cu/CeO₂ catalysts in which Cu²⁺ is dispersed on the surface of the CeO₂ (P. Bera et al. J. Catalysis (1999) 186:36-44) as<100 Å crystallites. That catalyst was active for catalyzing the reduction of NO by NH₃, CO reduction by NH₃, and hydrocarbon oxidation by NO.

Although significant advances have been made in the development of catalysts and processes for producing synthesis gas, there continues to be a need for more efficient and economical processes and catalysts that are capable of operating at moderate temperatures, are physically and chemically stable on stream and resist coking. Ideal syngas catalysts will also retain a high level of conversion activity and selectivity to carbon monoxide and hydrogen under conditions of high gas space velocity and elevated pressure and temperature for long periods of time on-stream.

### SUMMARY OF PREFERRED EMBODIMENTS

A method of synthesizing thermally stable catalysts for the production of synthesis gas is provided which employs combustion of the catalyst precursor materials and a combustible organic compound. Through this combustion process, the active catalytic components are anchored into the metal oxide support with a high degree of dispersion to provide fine particle, high surface area catalysts that overcome the drawbacks of many of the catalysts that are typically used for the production of syngas. The high surface area together with the high metal dispersion provide the desired active sites for the fast, selective oxidation of methane to syngas. Also, by anchoring the active phase onto the surface of thermally stable metal oxide supporting materials can prevent the active sites from sintering. As a result, ultrafine, high surface area catalysts prepared by the herein described combustion method are active, selective and stable for syngas production.

In accordance with certain embodiments of the present invention, a method of making a catalyst that is active for catalyzing the conversion of methane and oxygen to a product gas mixture comprising CO and H₂ under catalytic partial oxidation promoting conditions is provided. In certain embodiments, the method comprises combining (a) at least one decomposable precursor compound of a transition metal or metal oxide chosen from the group consisting of Rh, Ru, Pd, Pt, Au, Ag, Os, Ir, Sc, Ti, V, Cr, Mn, Fe, Co, Ni, Cu, Zr, Nb, Mo, Tc, Hf, Ta, W and Re, and oxides thereof, (b) at least one decomposable precursor compound of a base metal oxide chosen from the group consisting of the oxides of Be, Mg, Ca, Sr, Ba, Ra, Al, Ga, In, Tl, Si, Ge, Sn and Pb, (c) at least one combustible organic compound, for example, an amine, a hydrazide, urea or glycol, (d) a liquid mixing agent, and, (e) optionally, at least one decomposable precursor compound of a rare earth metal or metal oxide chosen from the group consisting of La, Ce, Pr, Nd, Pm, Sm, Eu, Gd, Tb, Dy, Ho, Er, Tm, Yb, Lu, Sc, Y and Th, and oxides thereof, such that a mixture is formed. The method further includes heating the mixture in the presence of O₂ until the mixture or combustible organic component thereof ignites, whereby a combustion residue is produced; optionally, the residue is calcined, preferably according to a predetermined heating program in an O₂-containing atmosphere, to yield a calcined combustion residue. In some embodiments, the heating program includes heating the residue, at a rate up to about 110° C./min, to a temperature in the range of 300-700° C. The method may also include heating the calcined combustion residue under reducing conditions, to provide a supported catalyst that is active for catalyzing the conversion of methane and oxygen to a product gas mixture comprising CO and H₂ under catalytic partial oxidation promoting conditions. In some embodiments the method also includes heating the calcined residue at a temperature within the operating range of a catalytic partial oxidation syngas production reactor, such as a temperature in the range of 600-2,000° C. In preferred embodiments of the catalyst making method, the liquid mixing agent is evaporated from the mixture prior to autoignition of the combustible compound. In some embodiments a phase separation reducing agent, such as nitric acid, is added to the mixture.

According to other embodiments of the present invention, a catalyst comprising the product of the above-described process is provided. In preferred embodiments, the catalyst is characterized by having a dispersion of nanometer diameter range particles of the transition metal or metal oxide deposited on the base metal oxide. In certain embodiments the particles are 2 to 100 nm in diameter, preferably 3-10 nm, and in some embodiments the diameter is about 8 nm. In certain preferred embodiments, the catalyst has the general formula αAOₓ-βBO_{y}-γCO_{z} wherein (a) A is a precious metal chosen from the group consisting of Rh, Ru, Pd, Pt, Au, Ag, Os and Ir, or A is a transition metal chosen from the group consisting of Sc, Ti, V, Cr, Mn, Fe, Co, Ni, Cu, Zr, Nb, Mo, Tc, Hf, Ta, W and Re; (b) B is a rare earth metal chosen from the group consisting of La, Ce, Pr, Nd, Pm, Sm, Eu, Gd, Tb, Dy, Ho, Er, Tm, Yb, Lu, Sc, Y and Th; (c) C is a base metal chosen from the group consisting of Be, Mg, Ca, Sr, Ba, Ra, Al, Ga, In, Tl, Si, Ge, Sn and Pb; (d) O is oxygen; (e) α, β, γ are the relative molar ratios of A, B and C, respectively, and α=0-0.2; β=0-0.5; γ=0.5-1; and (f) x, y, z are the numbers determined by the valence requirements of the metals A, B, and C, respectively. In some embodiments, the B rare earth metal is absent. Some embodiments of the catalyst comprise dispersed Rh⁰ and/or Rh oxide nanoparticles deposited on a base metal oxide which is, preferably, α-Al₂O₃, ZrO₂, CeO₂ or MgO. Some embodiments of the catalyst comprise dispersed Rh⁰ and/or Rh oxide nanoparticles and dispersed Sm⁰ and/or Sm oxide deposited on the base metal oxide. Other embodiments of the catalyst comprise dispersed Ni⁰ and/or Ni oxide nanoparticles deposited on the base metal oxide. The catalyst can be in the form of a monolith or can be in the form of divided or discrete structures or particulates. The term “monolith” as used herein is any singular piece of material of continuous manufacture such as solid pieces of metal or metal oxide or foam materials or honeycomb structures. The terms “discrete” structures, as used herein, refer to supports in the form of divided materials such as granules, beads, pills, pellets, cylinders, trilobes, extrudates, spheres or other rounded shapes, or another manufactured configuration. Alternatively, the divided material may be in the form of irregularly shaped particles. Preferably at least a majority (i.e., >50%) of the particles or distinct structures have a maximum characteristic length (i.e., longest dimension) of less than six millimeters, preferably less than three millimeters. According to some embodiments, the divided catalyst structures have a diameter or longest characteristic dimension of about 1/100″ to ¼″ (about 0.25 mm to 6.35 mm). In other embodiments they are in the range of about 50 microns to 6 mm. In preferred embodiments, the catalyst has an enhanced meso/macro pore structure and a characteristic BET surface area of at least 5 m²/g.

In accordance with other embodiments of the present invention, a method of converting methane or natural gas and O₂ to a product gas mixture containing CO and H₂ is provided. The process comprises, in a reactor, contacting a reactant gas mixture containing methane or natural gas and an O₂ containing gas with a catalytically effective amount of a catalyst as described above, while maintaining net catalytic partial oxidation promoting conditions. In industrial-scale commercial embodiments, the combustion produced catalyst may be prepared in large batches or in an automated continuous production process. In some embodiments, the method includes passing a stream of the reactant gas mixture over the catalyst at a gas hourly space velocity of 20,000-100,000,000 h⁻¹, preferably 100,000-25,000,000 hr⁻¹, and maintaining a catalyst residence time of no more than about 200 milliseconds, preferably less than 50 milliseconds, more preferably under 20 milliseconds for each portion of reactant gas mixture contacting the catalyst. A contact time of 10 milliseconds or less is highly preferred. In some embodiments the method includes preheating the reactant gas mixture to about 30° C.-750° C. before contacting the catalyst. In some embodiments a combustible gas, preferably propane, is added to facilitate light off of the reaction. In preferred embodiments, autothermal net catalytic partial oxidation reaction promoting conditions are maintained, which can include (a) adjusting the concentrations of methane or natural gas and O₂ in the reactant gas mixture, (b) adjusting the space velocity of the reactant gas mixture, (c) adjusting the temperature of the methane or natural gas and/or the O₂ containing gas, and (d) adjusting the operating pressure of the reactor. In some embodiments, N₂ is included in the reactant gas mixture, as a diluent, for example. In some embodiments the temperature of the methane or natural gas and/or the O₂ containing gas is adjusted to 600-1,200° C. prior to contacting the catalyst. In some embodiments the operating pressure of the reactor is in excess of 100 kPa (about 1 atm) while contacting the catalyst, and up to about 32,000 kPa (about 320 atmospheres), preferably between 200-10,000 kPa (about 2-100 atm), and more preferably above 3 atm. In some embodiments the concentrations of methane or natural gas and O₂ in the reactant gas mixture are such that the carbon:oxygen molar ratio is about 1.25:1 to 3.3:1, preferably about 1.3:1 to 2.3:1, and more preferably 1.5:1 to about 2.3:1, especially the CPOX stoichiometric ratio of 2:1. In preferred embodiments the natural gas feed comprises at least about 80% methane by volume. These and other embodiments, features and advantages of the present invention will become apparent with reference to the following description and drawings.

### BRIEF DESCRIPTION OF THE DRAWINGS

FIG. 1 is a graph showing the pore surface area over the pore diameter range of a Rh/Al₂O₃ catalyst prepared in accordance with the present invention.

FIG. 2 is a graph showing the pore volume over the pore diameter range of the same catalyst as in FIG. 1.

FIGS. 3(a) and (b) are transmission electron micrographs (TEMs) of a representative fresh Rh/Al₂O₃ sample showing the general morphology and Rh dispersion in the catalyst.

FIG. 4 shows transmission electron micrographs of a spent Rh/Al₂O₃ catalyst, in which (a) is from the top portion of the catalyst bed, and (b) is from the bottom portion.

FIGS. 5(a) and (b) are high resolution transmission electron microscopy (HRTEM) images of the samples shown in FIGS. 4(a) and (b), respectively.

FIG. 6 shows the XRD pattern of a representative fresh Rh/Al₂O₃ catalyst.

FIG. 7 shows the XRD patterns of a representative fresh Rh/CeO₂ catalyst.

### DETAILED DESCRIPTION OF PREFERRED EMBODIMENTS

New, highly dispersed and thermally stable metal oxide supported noble metals and mixed oxide catalysts are prepared as described in the following Examples. These catalysts have the general formula αAOₓ-βBO_{y}-γCO_{z}, wherein

A is one of the precious metals Rh, Ru, Pd, Pt, Au, Ag, Os or Ir or is a transition metal chosen from the group consisting of Sc, Ti, V, Cr, Mn, Fe, Co, Ni, Cu, Zr, Nb, Mo, Tc, Ru, Rh, Pd, Ag, Hf, Ta, W, Re, Os, Ir, Pt and Au, preferably Co, Ni, Mn, V or Mo;

B is a rare earth metal La, Ce, Pr, Nd, Pm, Sm, Eu, Gd, Tb, Dy, Ho, Er, Tm, Yb, Lu, Sc, Y and Th, preferably La, Yb, Sm or Ce;

C is a base metal chosen from Group II (i.e., Be, Mg, Ca, Sr, Ba and Ra), III (i.e., Al, Ga, In, Tl) and IV (i.e., Si, Ge, Sn, Pb) elements of the Periodic Table of the Elements, preferably Mg, Ca, Al or Si,

O is oxygen;

α, β, γ are the relative molar ratios of each metal oxide and α=0-0.2; β=0-0.5; χ=0.5-1; and

x, y, z are numbers that represent the stoichiometric elemental amount of oxygen in the three phases, i.e., active metals/metal oxides, promoters of rare earth metal oxides, and support oxides. The numbers are determined by the valence requirements of the metals A, B, and C, respectively. Their value can be zero when the corresponding metal stays in the metallic states.

In this general formula, if component A is in metallic form, this general formula can be presented as αA⁰-βBO_{y}-γCO_{z}. Alternatively, the catalyst can have the general formula: αAOₓ-γCO_{z} when component B is not used. The codes, A, C, O, α, γ, x, z, etc. have the same meaning as described above. When component A is in metallic form, this general formula becomes αA⁰-γCO_{z}.

General Procedure for Catalyst Preparation.

The precursor compounds (e.g., thermally decomposable metal salts) of the desired metal oxides, a combustible organic compound (such as amines, hydrazides, urea, glycol and the like) and a small amount of a liquid mixing agent, preferably water, are combined to form a mixture. The mixture (preferably a paste) is heated in air, and then the temperature of the mixture is ramped or gradually increased. The mixture begins dehydrating at 100-300° C., and a uniform solution forms. If necessary in order to avoid phase separation during this stage, the pH of the solution is adjusted by adding a suitable phase separation preventing agent such as nitric acid. The acid can be added before ramping the temperature or can be added during dehydration. The temperature is then further increased to the autoignition point of the mixture (e.g., 200-500° C.), the mixture ignites, and the strong exothermic oxidation reaction of the organic compound heats the mixture to above 1,000° C. within a second. During the combustion, the organic compound is burnt and the metal precursor compounds decompose to form the corresponding metal oxides or metals. The combustion process is so fast that the compositional uniformity of the mixture before the dispersion is reserved in the resulted mixed metal/metal oxides. The type of organic compound, its concentration in the mixture, the temperature ramping rate, as well as the environmental temperature, etc., all have influence on the maximum flame temperature and hence the properties (phase structure, dispersion, stability, etc.) of the final product. For example, by increasing the content of flammable organic compound, the flame temperature can be increased, which increases the stability of the catalyst but may decrease its surface area. Therefore, the above parameters can be varied and optimized based on the desired catalytic performance.

Optionally, the residue resulting from the combustion is then calcined in air, preferably at about 300-700° C. to burn off any flammable residuals. Also optionally, to obtain the active syngas catalyst, the calcined sample is reduced in a gas mixture containing hydrogen, preferably at a temperature in the range of 300-700° C., to convert the active component from the oxide to its metallic state.

The catalysts prepared by this combustion technique are physically distinct from those prepared by conventional methods such as precipitation, impregnation or washcoating and which employ conventional thermal decomposition techniques.

### EXAMPLE 1

### Rh/Alumina

A catalyst containing 4 wt. % Rh in Al₂O₃ was prepared by combustion synthesis, as follows: 0.651 g RhCl₃.xH₂O (Aldrich), and 56.5 g Al(NO₃)₃ 9H₂O (Aldrich) were mixed and dissolved in about 50 ml deionized water. The weight percent (wt. %) of Rh is based on the total weight of the catalyst, including the support. 33.8 g oxalic dihydrazide (Aldrich) was added to the above solution to form a paste. This paste was stirred to uniform and then divided into four 100 ml porcelain evaporating dishes. The dishes containing the redox mixture were heated up on a hot plate by ramping the temperature at about 10° C./min to ignition temperature. Initially, the solution boiled and dehydrated. At around 240° C., the paste became a uniform, clear, yellowish solution. At the point of complete dehydration, the mixture ignited, burnt and yielded a fluffy solid product. This product is collected and calcined at 400° C. in air for 4 hours. The powder product was pressed, crushed and sieved to form 20-40 mesh granules to facilitate the catalytic performance test for syngas production.

Active catalyst was obtained by reducing the calcined sample in flowing H₂/N₂ (50/50 vol. %) at total flow rate of 300 ml/min for 2 hours while heated at 500° C. prior to evaluation of its physical characteristics and catalytic activity, as described below.

To demonstrate the thermal stability of this catalyst, a portion (about 2 grams) of this reduced catalyst was further calcined at 1,000° C. in flowing air (50 ml/min) for 2 hours. The calcined sample was characterized with TEM analysis, as described below.

In another, similar preparation RhCl₃.xH₂O was added after the formation of paste containing Al(NO₃)₃ and oxalic dihydrazide. This catalyst had similar properties to that prepared as described above, as indicated by transmission electron micrographs and the x-ray diffraction patterns of the catalysts.

### EXAMPLE 2

### Pt/Alumina

A procedure similar to that used in Example 1 was followed to prepare 4 wt. % Pt/Al₂O₃ sample, except that 8.4 g hydrogen hexahydroxyplatinate (IV) (H₂PtCl₆) solution (8 wt. % in water) (Aldrich) was used instead of the 0.651 g RhCl₃.xH₂O that was used in Example 1. The rest of procedure was the same as described in Example 1. Alternatively, the procedure for making a combustion derived Pt/Al₂O₃ catalyst described by Bera et al. (J. Mater. Chem. (1999) 9:1801-1805) can be used, the disclosure of which is hereby incorporated herein by reference.

### EXAMPLE 3

### Ni/Alumina

A sample containing 5 wt. % Ni on an alumina support was prepared as follows: 1.98 g Ni(NO₃)₃.6H₂O (Aldrich) and 55.89 g Al(NO₃)₃.9H₂O were dissolved in about 50 mL deionized water. 34 g oxalic dihydrazide (Aldrich) was added to form a green suspension. When the suspension was heated on a hot plate, the mixture turned to a clear solution, then to blue, then to a gray paste. The paste was further heated to dehydrate, ignite and combust, following the procedure described in Example 1. The rest of this preparation procedure is the same as described in Example 1.

### EXAMPLE 4

### Rh/ZrO₂

A sample of Rh supported on ZrO₂ was also prepared similar to the procedure used in Example 1, but substituting ZrO₂ for the Al₂O₃. In this example, 0.4068 g RhCl₃.xH₂O (Aldrich), 9.0141 g ZrO(NO₃)₃.xH₂O (Aldrich) and 6 g oxalic dihydrazide (Aldrich), 50 ml deionized water and 1 ml nitric acid (70% solution) (Aldrich) were made into a uniform paste and heated to combust, as described in Example 1. The remainder of the preparation procedure was as described in Example 1.

### EXAMPLE 5

### Rh/CeO₂

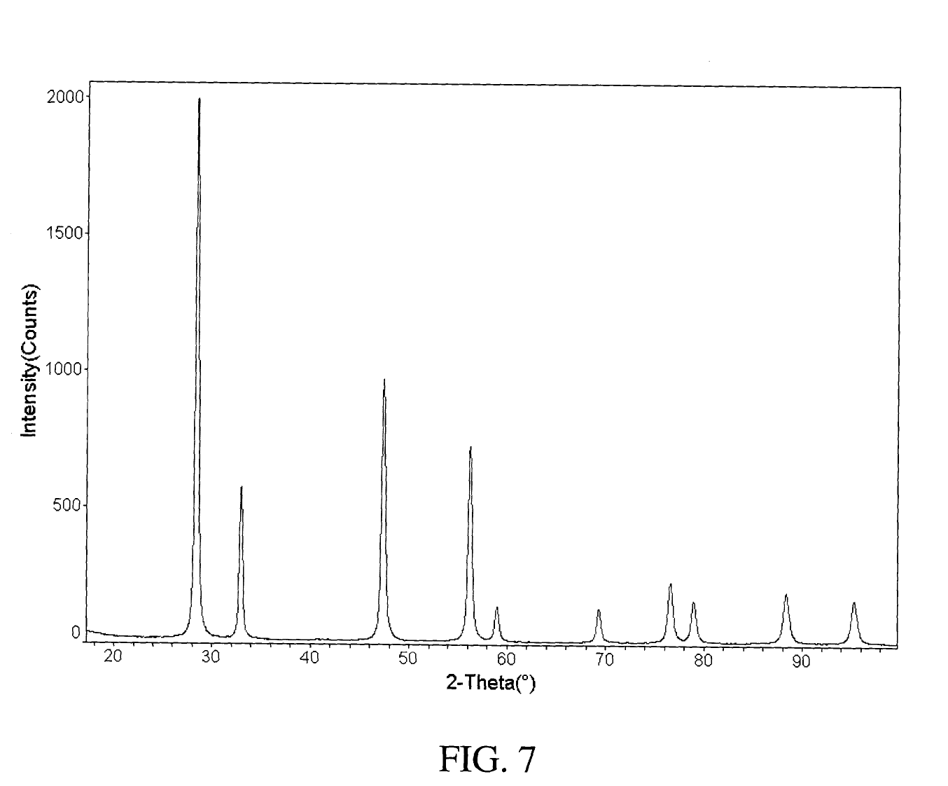

Using a procedure similar to that used in Example 1, but substituting CeO₂ for Al₂O₃, a sample containing 4 wt. % Rh carried on CeO₂ was obtained. 0.4066 g RhCl₃.xH₂O (Aldrich), 12.110 g Cerium (III) nitrate hexahydrate (Ce(NO₃)₃.6H₂O, Aldrich) and 6.1 g oxalic dihydrazide (Aldrich), 50 ml deionized water were made into a uniform paste and heated to combust as is described in Example 1. The rest of the preparation procedure was the same as used in Example 1. The XRD pattern of the resulting Rh/CeO₂ catalyst is shown in FIG. 7.

### EXAMPLE 6

### Rh/MgO

A similar procedure to that used for preparing Rh/Al₂O₃, described in Example 1, was used to prepare 4 wt. % Rh supported on MgO, substituting MgO for Al₂O₃. In this case, 0.651 g RhCl₃.xH₂O (Aldrich), 48.9 g magnesium nitrate hexahydrate (Mg(NO₃)₂.6H₂O, Aldrich) and 35 g oxalic dihydrazide (Aldrich), 50 ml deionized water and 1 ml nitric acid (70% solution) (Aldrich) were made into a uniform paste and heated to combust as is described in Example 1. The rest of the preparation procedure was the same as used in Example 1.

### EXAMPLE 7

### Rh—Sm/CeO₂

A sample of Rh and Sm supported on CeO₂ was prepared by following the same procedure as is described in Example 5, except 0.984 g Sm(III)(NO₃)₃.6H₂O was included in the combustion mixture. A catalyst with the nominal composition of 4 wt. % Rh/4 wt. % Sm/CeO2 was prepared.

Samples of the above-described catalyst preparations were examined using conventional transmission electron microscopy (TEM), powder x-ray diffraction (XRD) and BET/pore structure analysis techniques to assess their physical properties.

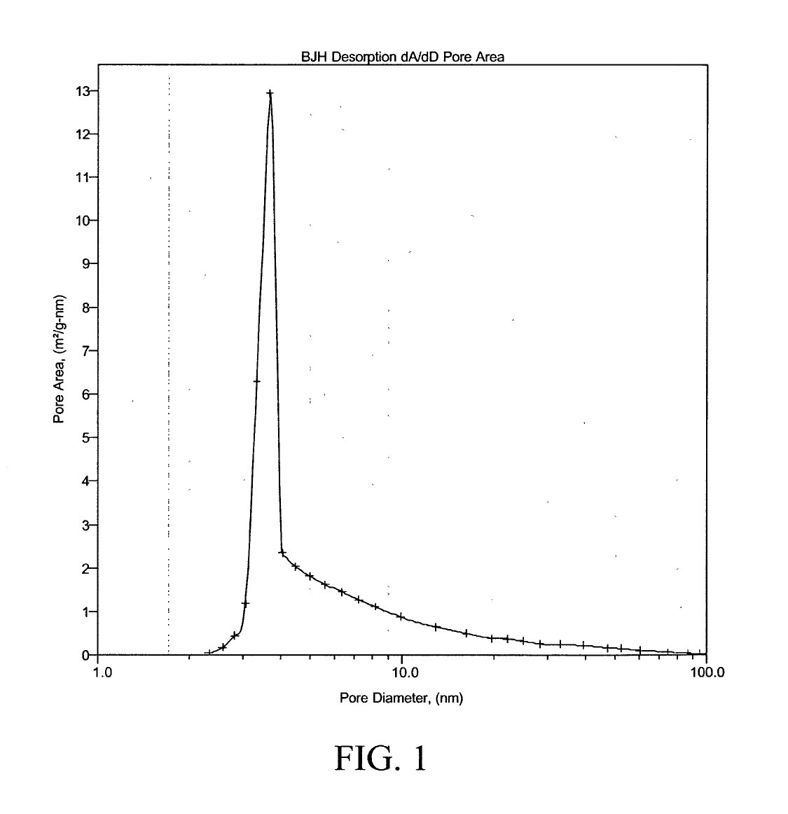

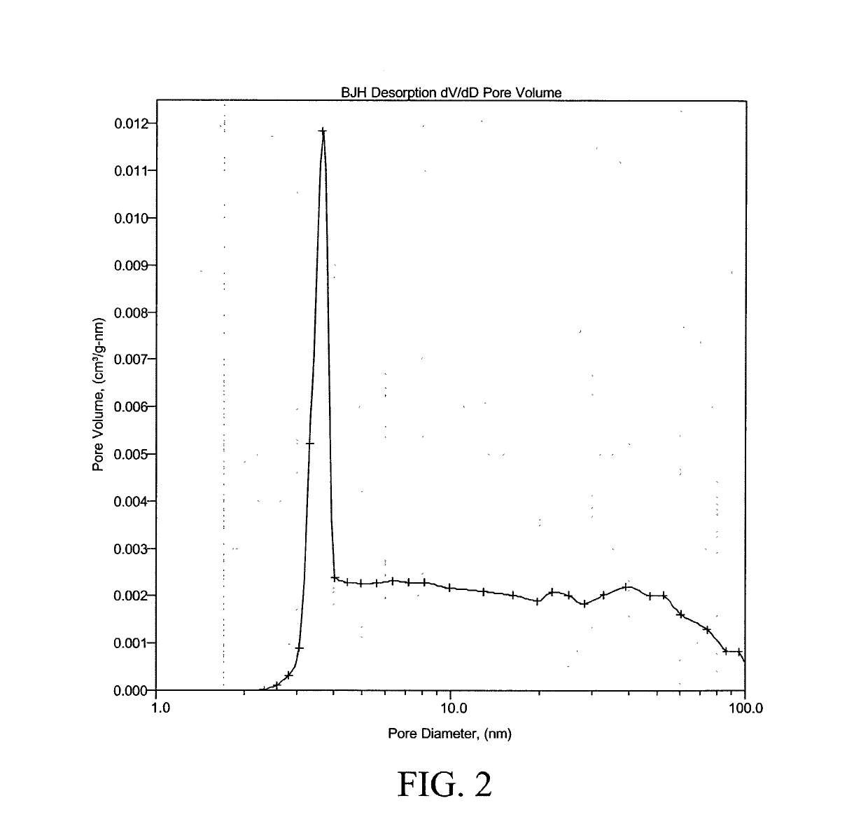

Surface area and pore structure. Each of the combustion-generated catalysts of Examples 1-7 are in the form of a fluffy powder. The Rh/alumina catalyst of Example 1 had a high surface area (27 m²/g) (BET) and a large pore structure (meso-pores in the range of 10-100 nm diameter). FIG. 1 shows the surface area distribution over the pore diameter range of a representative Rh/alumina catalyst prepared according to Example 1. FIG. 2 shows the pore volume over the pore diameter range of the same catalyst, as measured by BJH Desorption. The surface area of the pores in the range of 1.7-300 nm in diameter was 34 m²/g, as measured by BJH Desorption. The average pore diameter (4V/A) was 22 nm. It should be noted that the catalyst sample prepared using the present combustion technique has a unique pore structure, as shown in FIG. 1 and FIG. 2. It has a narrow pore distribution at pore size of about 3-4 nm which provides the catalyst with high surface area. This sample also has pores ranging from 4 nm to more than 100 nm. This unique pore distribution is especially advantageous for syngas catalysts. Preferably syngas production through selective oxidation of natural gas (catalytic partial oxidation or CPOX) is a short contact time reaction process, e.g., less than 200 milliseconds, preferably less than 50 milliseconds, and more preferably less than 20 milliseconds, with 10 milliseconds or under being highly preferred. In this process, the rate of reaction is typically strongly diffusion limited, that is, the active sites inside the micropores (i.e., <10 nm diameter) of a catalyst are hardly accessible to the reactant, and thus do not contribute appreciably to the overall reaction rate. The modified meso/macro pore structure, as is shown in FIG. 1 and FIG. 2, can decrease this diffusion limit by using the meso/macro pores with diameter of up to 100 nm as the diffusion channel for the reactant molecule to make all active sites accessible to the reactant. This special characteristic partially explains the high activity of these catalysts, as is shown below. Although it is preferred to use these catalysts for syngas production at contact times of less than 100 milliseconds, the process can also employ contact times longer than 100 milliseconds.

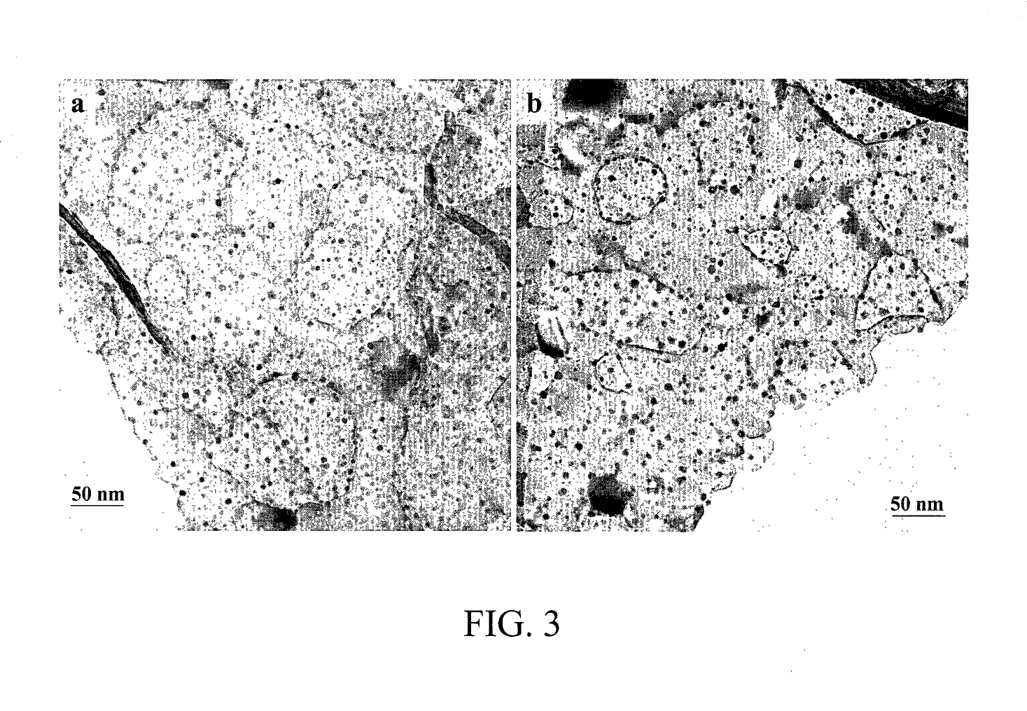

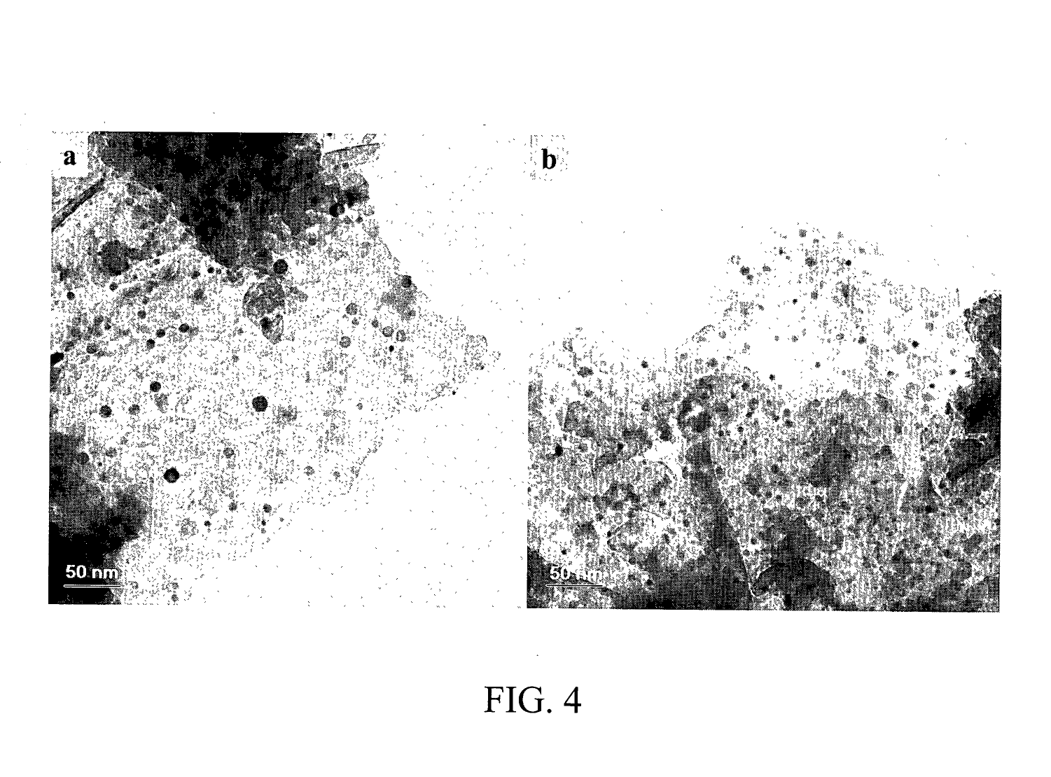

Metal dispersion and phase structure. When Rh is used as the precious metal, Rh is highly dispersed in the final catalyst, as can be seen in FIGS. 3-5. The metal particle size ranges from 2 to about 100 nm, and the average metal particle size (diameter or longest dimension) is preferably between about 3 and 100 nm, more preferably about 8 nm, which is much smaller than the Rh crystallites achieved by using a conventional precipitation or impregnation method. FIG. 3(a) and (b) are representative TEM micrographs of Rh/Al₂O₃ catalyst prepared as described in Example 1. FIG. 4 shows representative TEM micrographs of the spent Rh/Al₂O₃ catalyst sample showing that the general morphology is similar to the fresh catalyst and Rh is still in highly dispersed form in the top (a) and bottom (b) portions of the catalyst bed. The catalyst temperature reached as high as 1,200° C. during these particular syngas reactions. Comparing the TEM patterns of the fresh and spent samples, the TEM results shown in FIG. 4 indicate no sintering of rhodium occurred on the spent catalysts, and demonstrates the high thermal stability of catalyst samples generated from combustion preparation.

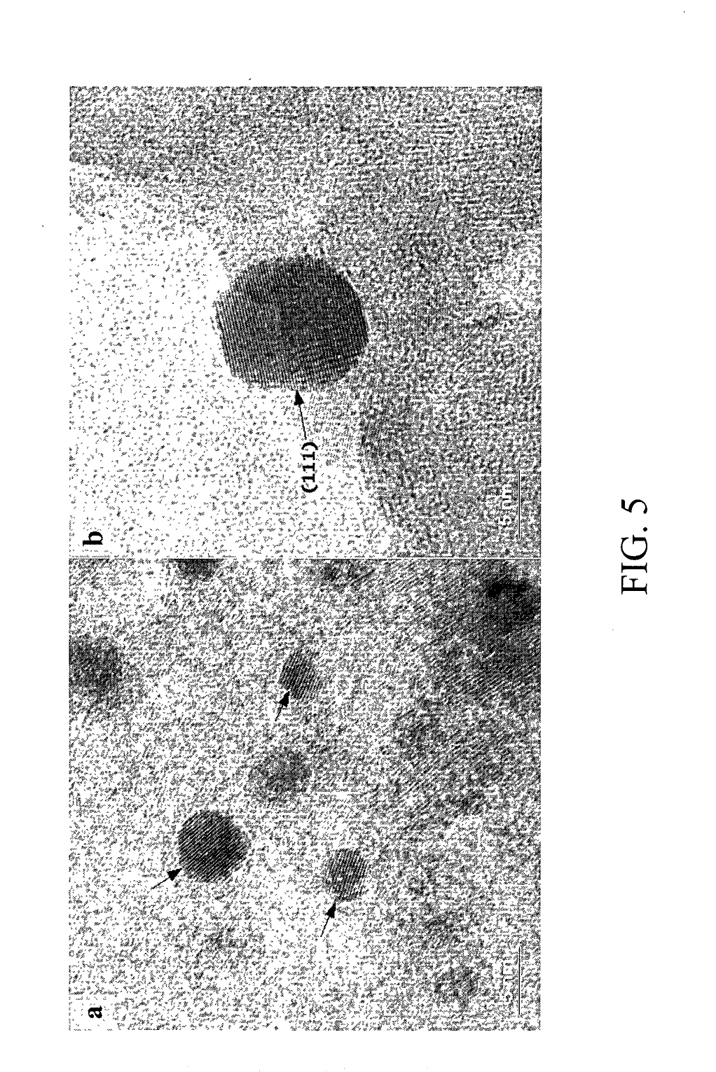

FIGS. 5(a) and (b) are high resolution transmission electron microscopy (HRTEM) images of a representative spent catalyst, Rh/Al₂O₃, prepared by the combustion method. Again, this result shows the particle sizes of Rh are in the range of 3-10 nm. It is also of significance that, on representative spent catalyst samples, there is no indication of the carbon deposition that is typically seen on spent catalysts that are prepared using conventional methods, such as impregnation, precipitation, etc. The arrows in FIGS. 5(a) and (b) indicate the Rh(111) lattice fringes corresponding to the (111) planes of Rh metal. Since these fringes are clearly visible in the TEMs, the absence of graphitic carbon overlayers on the exposed Rh metal surface of the Rh particles is apparent. These results clearly demonstrate the superior carbon-resistant of the syngas catalysts of this invention.

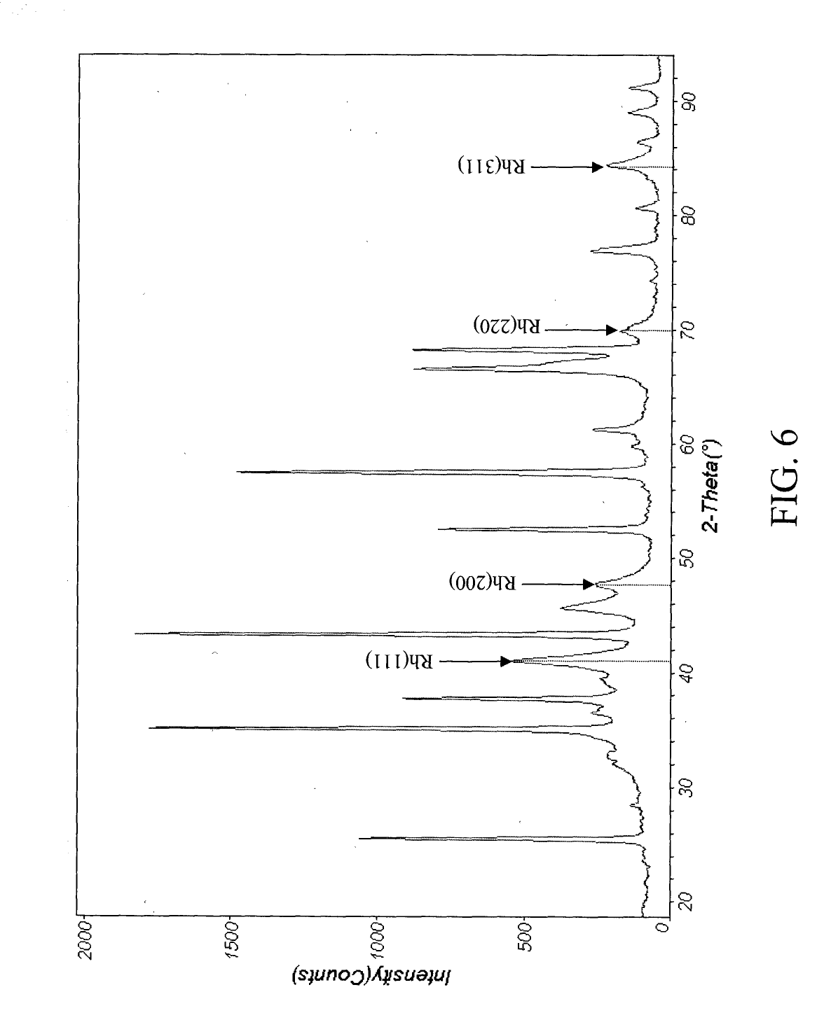

FIG. 6 shows the XRD pattern of a representative fresh Rh/Al₂O₃ catalyst sample, prepared as described in Example 1. The four characteristic Rh diffraction lines, Rh(111), Rh(200), Rh(220) and Rh(311), are highlighted. Each Rh line, Rh(111), Rh(200), Rh(220) or Rh(311), corresponds to one specific set of planes as represented by their Miller indices. The XRD pattern indicates that alpha alumina is the major crystalline phase having an average crystal size of 46 nm. This is a major factor in establishing the high surface area (27 m²/g) of this catalyst sample. The estimated Rh crystal size is 8 nm.

FIG. 7 shows the XRD pattern of freshly prepared Rh/CeO₂ prepared as described in Example 5. The average crystal size of CeO₂ is 27 nm. No Rh is seen by XRD in FIG. 7, and a TEM of the same sample indicated only occasional Rh particles (not shown).

It is preferable to size the particles or to press the powder catalyst obtained in the combustion synthesis into granules, beads, pills, pellets, cylinders, trilobes, extrudates, spheres or other rounded shapes, or other suitable shapes. A conventional catalyst binder material such as alumina, silica, graphite, fatty acid could be combined with the powder, if desired, to facilitate pelletization, using standard techniques that are well-known in the art. Preferably at least a majority (i.e., >50%) of the particles or distinct structures have a maximum characteristic length (i.e., longest dimension) of less than six millimeters, preferably less than three millimeters. According to some embodiments, the divided catalyst structures have a diameter or longest characteristic dimension of about 1/100″ to ¼″ (about 0.25 mm to 6.35 mm). In other embodiments they are in the range of about 50 microns to 6 mm.

The combustion generated catalyst powders are also suitable for combining with an appropriate carrier, such as a base metal oxide, preferably a refractory base metal oxide, and extruding or forming the catalyst suspension into a three-dimensional structured catalyst, such as a foam monolith. Alternatively, the powder catalyst may be suspended in a suitable carrier and washcoated onto a preformed honeycomb or other monolith support. The catalyst can be structured as, or supported on, a refractory oxide “honeycomb” straight channel extrudate or monolith, or other configuration having longitudinal channels or passageways permitting high space velocities with a minimal pressure drop. Such configurations are known in the art and described, for example, in Structured Catalysts and Reactors, A. Cybulski and J. A. Moulijn (Eds.), Marcel Dekker, Inc., 1998, p. 599-615 (Ch. 21, X. Xu and J. A. Moulijn, “Transformation of a Structured Carrier into Structured Catalyst”), which is hereby incorporated herein by reference.

Test Procedure

Representative catalysts prepared as described in the foregoing Examples were evaluated for their ability to catalyze the partial oxidation reaction in a conventional flow apparatus with a 19 mm O.D.×13 mm I.D. quartz insert embedded inside a refractory-lined steel vessel. The quartz insert contained the catalyst packed between two foam disks. Both disks typically consisted of 80-ppi zirconia-toughened alumina. Preheating the methane or natural gas that flowed through the catalyst system provided the heat needed to start the reaction. Oxygen was mixed with the methane or natural gas immediately before the mixture entered the catalyst system. The methane or natural gas was spiked with propane as needed to initiate the partial oxidation reaction, then the propane was removed as soon as ignition occurred. Once the partial oxidation reaction commenced, the reaction proceeded autothermally. Two Type K thermocouples with ceramic sheaths were used to measure catalyst inlet and outlet temperatures. The molar ratio of CH₄ to O₂ was generally about 2:1, however the relative amounts of the gases, the catalyst inlet temperature and the reactant gas pressure could be varied by the operator according to the particular parameters being evaluated. The product gas mixture was analyzed for CH₄, O₂, CO, H₂, CO₂ and N₂ using a gas chromatograph equipped with a thermal conductivity detector. A gas chromatograph equipped with flame ionization detector analyzed the gas mixture for CH₄, C₂H₆, C₂H₄ and C₂H₂. The CH₄ conversion levels and the CO and H₂ product selectivities obtained for each catalyst evaluated in this test system are considered predictive of the conversion and selectivities that will be obtained when the same catalyst is employed in a commercial scale short contact time reactor at least under similar conditions of reactant concentrations, temperature, reactant gas pressure and space velocity.

The performance of the representative catalysts in catalyzing the production of synthesis gas at 1 atm pressure is shown in Table 1. The Rh/Al₂O₃ catalyst listed in Table 1 contains 4 wt. % Rh on alumina and was prepared as described in Example 1. This catalyst was also tested at high pressure (about 3 atm) with high gas hourly space velocity, and the results are shown in Table 2. Table 1 also shows the performance of a Rh/MgO catalyst prepared as described in Example 6. These results demonstrate that the new catalysts prepared using the above-described combustion synthesis methods can produce syngas at short contact time with high activity and high selectivity for CO and H₂ products.

TABLE 1
Catalytic Performance of Representative Catalysts Under Atmospheric Pressure

| | Catalyst | | CH₄ | CO | H₂ | CO₂ | WHSV | | |
|---|---|---|---|---|---|---|---|---|---|
| Catalyst | Weight | Flow Rate | Conversion | Selectivity | Selectivity | Selectivity | (ml/(g cat. | T*ₒᵤₜ | |
| formula | (g) | (ml/min) | (%) | (%) | (%) | (%) | hour) | (C) | ΔP (psi) |
| Rh/Al₂O₃ | 2 | 3500 | 91 | 94.9 | 92.0 | 5 | 105000 | 669 | 2 |
| Rh/Al₂O₃ | 2 | 5000 | 92.7 | 95.9 | 92.6 | 4 | 150000 | 708 | 2.8 |
| Rh/Al₂O₃ | 1 | 3500 | 94.3 | 96.5 | 93.3 | 3.4 | 210000 | 743 | 1.7 |
| Rh/Al₂O₃ | 1 | 5000 | 94.4 | 96.9 | 93.2 | 3 | 300000 | 765 | 2.6 |
| Rh/Al₂O₃ | 0.5 | 3500 | 94.5 | 96.6 | 93.5 | 3.4 | 420000 | 720 | 0.7 |
| Rh/Al₂O₃ | 0.5 | 5000 | 94.8 | 97 | 93.5 | 3 | 600000 | 748 | 1 |
| Rh/MgO | 0.8 | 3500 | 94.4 | 96.6 | 93.4 | 3.4 | 262500 | 702 | 1.3 |
| Rh/MgO | 0.8 | 5000 | 94.6 | 96.9 | 93.4 | 3.1 | 375000 | 727 | 1.8 |

Note:
Other reaction conditions are Preheat = 300° C. and oxygen to methane molar ratio equals to 0.55

Definitions:

$${CH}_{4} conversion ( \% ) \text{:} X ( {CH}_{4} ) = \frac{\sum ( [ Ci ] \cdot n ) - [ {CH}_{4} ]}{\sum ( [ Ci ] \cdot n )} \times 100 \%$$

$$CO selectivity ( \% ) \text{:} S ( CO ) = \frac{[ CO ]}{\sum ( [ Ci ] \cdot n ) - [ {CH}_{4} ]} \times 100 \%$$

$${CO}_{2} selectivity ( \% ) \text{:} S ( CO ) = \frac{[ {CO}_{2} ]}{\sum ( [ Ci ] \cdot n ) - [ {CH}_{4} ]} \times 100 \%$$

$$Hydrogen selectivity ( \% ) \text{:} S ( {H}_{2} ) = \frac{\frac{1}{2} \times {[ {H}_{2} ]}_{produced}}{{[ {CH}_{4} ]}_{in} - {[ {CH}_{4} ]}_{out}} \times 100 \%$$

Where, [CH₄], methane molar flow in the product; [Ci], molar flow of component i in the product; n is the number of carbon in component i. [CO], molar flow of CO in the product.

WHSV: (weight hourly space velocity, ml/(gCat.hr)) is used to describe the catalyst space yield. It is calculated by dividing total hourly feed flow rate at standard condition (ml/hr) by the total weight of catalyst (gcat)

TABLE 2
Catalytic Performance of Representative Catalysts under 45 psi

| Catalyst | Catalyst | CH₄ | CO | H₂ | CO₂ | WHSV | T*ₒᵤₜ | |
|---|---|---|---|---|---|---|---|---|
| formula | Weight (g) | Conversion (%) | Selectivity (%) | Selectivity (%) | Selectivity (%) | (mL/(gCat. hour) | (C) | ΔP (psi) |
| Rh/Al₂O₃ | 1 | 95 | 96 | 92.0 | 4 | 2,000,000 | 810 | 4 |

Note:
Other reaction conditions are Preheat = 300° C. and oxygen to methane mass ratio equals to 1.05 to 1.
Definitions: the same as for Table 1.

Process of Producing Syngas

A feed stream comprising a light hydrocarbon feedstock and an O₂-containing gas is contacted with one of the above-described combustion deposited metal-metal oxide catalysts, which is active for catalyzing the efficient conversion of methane or natural gas and molecular oxygen to primarily CO and H₂ by a net catalytic partial oxidation (CPOX) reaction. Preferably a very fast contact (i.e., millisecond range)/fast quench (i.e., less than one second) reactor assembly is employed. Several schemes for carrying out catalytic partial oxidation (CPOX) of hydrocarbons in a short contact time reactor are well known and have been described in the literature. The reactor is essentially a tube made of materials capable of withstanding the temperatures generated by the exothermic CPOX reaction (reaction 2, above). The reactor includes feed injection openings, a mixing zone, a reaction zone containing a catalyst, and a cooling zone. In a fixed-bed configuration, thermal radiation shields or barriers are preferably positioned immediately upstream and downstream of the catalyst bed in a fixed-bed configuration. In commercial scale operations the reactor may be constructed of, or lined with, a refractory material that is capable of withstanding the temperatures generated by the CPOX reaction.

The light hydrocarbon feedstock may be any gaseous hydrocarbon having a low boiling point, such as methane, natural gas, associated gas, or other sources of C₁-C₅ hydrocarbons. The hydrocarbon feedstock may be a gas arising from naturally occurring reserves of methane which contain carbon dioxide. Preferably, the feed comprises at least 50% by volume methane, more preferably at least 75% by volume, and most preferably at least 80% by volume methane. The hydrocarbon feedstock is in the gaseous phase when contacting the catalyst. The hydrocarbon feedstock is contacted with the catalyst as a mixture with an O₂-containing gas, preferably pure oxygen. The oxygen-containing gas may also comprise steam and/or CO₂ in addition to oxygen. Alternatively, the hydrocarbon feedstock is contacted with the catalyst as a mixture with a gas comprising steam and/or CO₂.

For the purposes of this disclosure, the term “net catalytic partial oxidation reaction” means that the CPOX reaction (Reaction 2) predominates. However, other reactions such as steam reforming (see Reaction 1), dry reforming (Reaction 5) and/or water-gas shift (Reaction 6) may also occur to a lesser extent.

CH₄+CO₂⇄2CO+2H₂ (5)

CO+H₂O⇄CO₂+H₂ (6)

The relative amounts of the CO and H₂ in the reaction product mixture resulting from the net catalytic partial oxidation of the methane or natural gas and oxygen feed mixture are preferably about 2:1H₂:CO, like the stoichiometric amounts of H₂ and CO produced in the partial oxidation reaction of Reaction 2.

As the preheated feed gas mixture passes over the catalyst to the point at which they ignite, an autothermal net catalytic partial oxidation reaction ensues. Preferably, the reaction conditions are maintained to promote continuation of the autothermal net catalytic partial oxidation process. For the purposes of this disclosure, “autothermal” means that after catalyst ignition, no additional heat must be supplied to the catalyst in order for the production of synthesis gas to continue. Autothermal reaction conditions are promoted by optimizing the concentrations of hydrocarbon and O₂ in the reactant gas mixture preferably within the range of about a 1.5:1 to about 2.3:1 ratio of carbon:oxygen. The hydrocarbon:oxygen ratio is the most important variable for maintaining the autothermal reaction and the desired product selectivities. Pressure, residence time, amount of feed preheat and amount of nitrogen dilution, if used, also affect the reaction products. All of these variables are preferably adjusted as necessary such that the desired H₂:CO ratio is achieved in the syngas emerging from the reactor. In some situations steam is also included in the reactant gas mixture, such as when it is desirable to produce extra hydrogen and/or to control the outlet temperature. The ratio of steam to carbon by weight ranges from 0 to 1. Preferably, the methane-containing feed and the oxygen-containing gas are mixed in such amounts to give a carbon (i.e., carbon in methane) to oxygen (i.e., molecular oxygen) ratio from about 1.5:1 to about 3.3:1, more preferably, from about 1.7:1 to about 2.1:1, and especially the stoichiometric ratio of 2:1. In some situations, such as when the methane-containing feed is a naturally occurring methane reserve, carbon dioxide may also be present in the methane-containing feed without detrimentally affecting the process. Depending on the particular situation, it may also be desirable at times to adjust the concentrations of the reactant gas mixture in order to increase or decrease the exothermicity of the process, maintain autothermal and enhance production of CO and H₂ at the desired ratio. The process is preferably operated at catalyst temperatures of from about 600° C. to about 2,000° C., preferably up to about 1,600° C. The hydrocarbon feedstock and the oxygen-containing gas are preferably pre-heated at a temperature between about 30° C. and 750° C. before contact with the catalyst to facilitate light-off of the reaction.

The process is operated at atmospheric or superatmospheric pressures, the latter being preferred. The pressures may be from about 100 kPa to about 32,000 kPa (about 1-320 atm), preferably from about 200 kPa to 10,000 kPa (about 2-100 atm).

The hydrocarbon feedstock and the oxygen-containing gas may be passed over the catalyst at any of a variety of space velocities. Space velocities for the process, stated as gas hourly space velocity (GHSV), are from about 20,000 to about 100,000,000 hr⁻¹, preferably from about 100,000 to about 25,000,000 hr⁻¹. Although for ease in comparison with prior art systems space velocities at standard conditions have been used to describe the present invention, it is well recognized in the art that residence time is the inverse of space velocity and that the disclosure of high space velocities equates to low residence times on the catalyst. Under these operating conditions a flow rate of reactant gases is maintained sufficient to ensure a residence time of no more than 200 milliseconds, preferably less than 50 milliseconds, and more preferably under 20 milliseconds with respect to each portion of reactant gas in contact with the catalyst system. A residence time of 10 milliseconds or less is highly preferred.

For commercial scale production of synthesis gas the above-described catalyst compositions may be more efficiently and economically prepared in large quantities and/or continuously produced as described in co-pending U.S. patent application Ser. No. 10/184,473, the disclosure of which is incorporated hereby by reference. Briefly described, the catalyst is prepared by a method comprising (a) combining in a mixing vessel at least one decomposable precursor compound of a catalytically active metal or metal oxide, (b) optionally, at least one decomposable precursor compound of a refractory metal oxide support, (c) at least one combustible organic compound, and, (d) optionally, a liquid mixing agent, to form a mixture. The mixture is introduced into an evaporator and the liquid mixing agent, if present, is evaporated and/or a portion of the combustible organic compound is evaporated, so that a catalyst intermediate results. The catalyst intermediate is introduced into a furnace and heated to the point of autoignition and allowed to combust, yielding a combustion product. The product of combustion may then be calcined before further processing. Additional catalyst processing can include, in a shaping unit, forming the combustion product into a predetermined shape. The shaped catalyst may then be treated in an activation unit to heating in a reducing atmosphere, or other activating conditions, to provide a larger quantity or a continuous supply of the activated catalyst for use in an industrial-scale reactor for large-scale output of synthesis gas.

The product gas mixture emerging from the reactor is harvested and may be routed directly into any of a variety of applications. One such application for the CO and H₂ product stream is for producing higher molecular weight hydrocarbon compounds using Fischer-Tropsch technology. It is an advantage of the present process that efficient syngas production at superatmospheric operating pressure facilitates the direct transition to a downstream process, such as a Fischer-Tropsch process, oftentimes without the need for intermediate compression. Alternatively, the syngas product can serve as a source of H₂ for fuel cells, in which case one of the above-described catalysts that provides enhanced selectivity for H₂ product may be selected, and process variables can be adjusted such that a H₂:CO ratio greater than 2:1 may be obtained, if desired. Fuel cells are chemical power sources in which electrical power is generated in a chemical reaction. The most common fuel cell is based on the chemical reaction between a reducing agent such as hydrogen and an oxidizing agent such as oxygen.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. The disclosures of all patents, patent applications and publications cited above are hereby incorporated herein by reference. The discussion of certain references in the Description of Related Art is not an admission that they are prior art to the present invention, especially any references that may have a publication date after the priority date of this application.

What is claimed is:

1. A method of making a catalyst that is active for catalyzing the conversion of methane and oxygen to a product gas mixture comprising CO and H₂ under catalytic partial oxidation promoting conditions, said method comprising:
combining
at least one decomposable precursor compound of a transition metal or metal oxide chosen from the group consisting of Rh, Ru, Pd, Pt, Au, Ag, Os, Ir, Sc, Ti, V, Cr, Mn, Fe, Co, Ni, Cu, Zr, Nb, Mo, Tc, Hf, Ta, W and Re, and oxides thereof,
at least one decomposable precursor compound of a base metal oxide chosen from the group consisting of the oxides of Be, Mg, Ca, Sr, Ba, Ra, B, Al, Ga, In, Ti, C, Si, Ge, Sn and Pb,
at least one combustible organic compound,
optionally, a liquid mixing agent, and,
optionally, at least one decomposable precursor compound of a rare earth metal or metal oxide chosen from the group consisting of La, Ce, Pr, Nd, Pm, Sm, Eu, Gd, Tb, Dy, Ho, Er, Tm, Yb, Lu, Sc, Y and Th, and oxides thereof, such that a mixture is formed;
heating said mixture in the presence of O₂ until said mixture or combustible organic component thereof ignites, whereby a combustion residue is produced;
optionally, calcining said residue to yield a calcined combustion residue;
optionally, heating said calcined residue at a temperature within the operating range of a catalytic partial oxidation syngas production reactor; and
optionally, heating said calcined combustion residue under reducing conditions, to provide a supported catalyst that is active for catalyzing the conversion of methane and oxygen to a product gas mixture comprising CO and H₂ under catalytic partial oxidation promoting conditions.

2. The method of claim 1 wherein said calcining comprises heating said residue according to a predetermined heating program in an O₂-containing atmosphere.

3. The method of claim 2 wherein said predetermined heating program includes heating the combustion residue at rate up to about 10° C./min to a temperature in the range of 300-700° C.

4. The method of claim 1 wherein said optional calcining comprises heating the combustion residue to a temperature in the range of 600-2,000° C.

5. The method of claim 1 comprising evaporating said liquid mixing agent from said mixture prior to said ignition.

6. The method of claim 1 further comprising adding a phase separation reducing agent to said mixture.

7. The method of claim 1 wherein said transition metal or metal oxide is chosen from the group consisting of Rh, Ru, Pd, Pt, Au, Ag, Os and Ir, and oxides thereof.

8. The method of claim 1 wherein said transition metal or metal oxide is chosen from the group consisting of Co, Ni, Mn, V and Mo, and oxides thereof.

9. The method of claim 1 wherein said base metal oxide is chosen from the group consisting of the oxides of Mg, Ca, Al and Si.

10. The method of claim 1 wherein said rare earth metal or metal oxide is chosen from the group consisting of La, Yb, Sm, Ce and oxides thereof.

11. The method of claim 1 wherein said combustible organic compound is chosen from the group consisting of amines, hydrazides, urea and glycol.

12. A catalyst comprising the product of the method of claim 1.

13. The catalyst of claim 12 wherein said catalyst comprises a dispersion of nanometer diameter range particles of said transition metal or metal oxide deposited on said base metal oxide.

14. The catalyst of claim 13 wherein said particles of precious metal or metal oxide or said transition metal or metal oxide are 2 to 100 nm in diameter.

15. The catalyst of claim 14 wherein said particles of precious metal or metal oxide or said transition metal or metal oxide have an average particle diameter of between 3 and 10 nm.

16. The catalyst of claim 15 wherein said particles of precious metal or metal oxide or said transition metal or metal oxide have an average particle diameter of 8 nm.

17. The catalyst of claim 12 having the general formula αAOₓ-βBO_{y}-γCO_{z} wherein
A is a precious metal chosen from the group consisting of Rh, Ru, Pd, Pt, Au, Ag, Os and Ir, or A is a transition metal chosen from the group consisting of Sc, Ti, V, Cr, Mn, Fe, Co, Ni, Cu, Zr, Nb, Mo, Tc, Hf, Ta, W and Re;
B is a rare earth metal chosen from the group consisting of La, Ce, Pr, Nd, Pm, Sm, Eu, Gd, Tb, Dy, Ho, Er, Tm, Yb, Lu, Sc, Y and Th;
C is a base metal chosen from the group consisting of Be, Mg, Ca, Sr, Ba, Ra, Al, Ga, In, Tl, Si, Ge, Sn and Pb;
O is oxygen;
α, β, γ are the relative molar ratios of A, B and C, respectively, and α=0-0.2; β=0-0.5; γ=0.5-1; and
x, y, z are the numbers determined by the valence requirements of the metals A, B, and C, respectively.

18. The catalyst of claim 12 having the general formula αAOₓ-γCO_{z} wherein
A is a precious metal chosen from the group consisting of Rh, Ru, Pd, Pt, Au, Ag, Os and Ir or A is a transition metal chosen from the group consisting of Sc, Ti, V, Cr, Mn, Fe, Co, Ni, Cu, Zr, Nb, Mo, Tc, Ru, Rh, Pd, Ag, Hf, Ta, W, Re, Os, r, Pt and Au;
C is a base metal chosen from the group consisting of Be, Mg, Ca, Sr, Ba and Ra, Al, Ga, In, Ti, Si, Ge, Sn and Pb;
O is oxygen;
α, γ are the relative molar ratios of A and C, respectively, α=0-0.2; γ=0.5-1; and
x and z are the numbers determined by the valence requirements of the metals A and C, respectively.

19. The catalyst of claim 12 comprising dispersed Rh⁰ and/or Rh oxide nanoparticles deposited on a base metal oxide chosen from the group consisting of α-Al₂O₃, ZrO₂, CeO₂ and MgO.

20. The catalyst of claim 12 comprising dispersed Rh⁰ and/or Rh oxide nanoparticles and dispersed Sm⁰ and/or Sm oxide deposited on a base metal oxide chosen from the group consisting of α-Al₂O₃, ZrO₂, CeO₂ or MgO.

21. The catalyst of claim 12 comprising dispersed Ni⁰ and/or Ni oxide nanoparticles deposited on a base metal oxide chosen from the group consisting of α-Al₂O₃, ZrO₂, CeO₂ and MgO.

22. The catalyst of claim 12 comprising a monolith structure.

23. The catalyst of claim 12 comprising a particulate structure.

24. The catalyst of claim 23 wherein said particulate structure is chosen from the group consisting of particles, granules, beads, pills, pellets, cylinders, trilobes, extrudates, spheres or other rounded shapes.

25. The catalyst of claim 23 wherein said particulate structure has a diameter or longest characteristic dimension of about 1/100″ to ¼″ (about 0.25 mm to 6.35 mm)

27. The catalyst of claim 23 wherein said particulate structure has a diameter or longest characteristic dimension in the range of about 50 microns to 6 mm.

28. A catalyst for the production of synthesis gas, said catalyst containing:
up to 0.2 relative molar ratio of a metal chosen from the group consisting of Rh, Ru, Pd, Pt, Au, Ag, Os, Ir, Sc, Ti, V, Cr, Mn, Fe, Co, Ni, Cu, Zr, Nb, Mo, Tc, Hf, Ta, W and Re;
optionally, up to 0.5 relative molar ratio of a rare earth metal chosen from the group consisting of La, Ce, Pr, Nd, Pm, Sm, Eu, Gd, Tb, Dy, Ho, Er, Tm, Yb, Lu, Sc, Y and Th; and
at least 0.5 relative molar ratio of a base metal oxide chosen from the group consisting of the oxides of Be, Mg, Ca, Sr, Ba and Ra, Al, Ga, In, Tl, Si, Ge, Sn and Pb, said catalyst having a modified meso/macro pore structure, a characteristic metal dispersion of said transition metal on said base metal oxide of at least 5 m²/g (BET), and having activity for catalyzing the conversion of methane and oxygen to a product gas mixture comprising CO and H₂ under catalytic partial oxidation promoting conditions.

29. A method of converting methane or natural gas and O₂ to a product gas mixture containing CO and H₂, the process comprising, in a reactor, contacting a reactant gas mixture containing methane or natural gas and an O₂ containing gas with a catalytically effective amount of the catalyst of claim 12 under net catalytic partial oxidation promoting conditions.

30. The method of claim 29 wherein said contacting comprises passing a stream of said reactant gas mixture over said catalyst at a gas hourly space velocity of at least about 20,000 h⁻¹.

31. The method of claim 29 wherein said step of contacting comprises passing said reactant gas mixture over said catalyst at a gas hourly space velocity up to 100,000,000 h⁻¹.

32. The method of claim 29 comprising passing said reactant gas mixture over said catalyst at a gas hourly space velocity in the range of 100,000-25,000,000 hr⁻¹.

33. The method of claim 29 wherein said contacting comprises maintaining a catalyst residence time of no more than about 200 milliseconds for each portion of reactant gas mixture contacting said catalyst.

34. The method of claim 33 wherein said contact time is less than 50 milliseconds.

35. The method of claim 34 wherein said contact time is less than 20 milliseconds.

36. The method of claim 35 wherein said contact time is 10 milliseconds or less.

37. The method of claim 29 comprising preheating said reactant gas mixture to about 30° C.-750° C. before contacting said catalyst.

38. The method of claim 29 comprising adding propane or other combustible gas to said reactant gas mixture sufficient to initiate a net catalytic partial oxidation reaction.

39. The method of claim 29 comprising maintaining autothermal net catalytic partial oxidation reaction promoting conditions.

40. The method of claim 39 wherein said step of maintaining autothermal net catalytic partial oxidation reaction promoting conditions comprises:
adjusting the concentrations of methane or natural gas and O₂ in said reactant gas mixture,
adjusting said space velocity of said reactant gas mixture,
adjusting the temperature of said methane or natural gas and/or said O₂ containing gas, and
adjusting the operating pressure of said reactor.

41. The method of claim 39 comprising including N₂ in said reactant gas mixture.

42. The method of claim 39 comprising including steam in said reactant gas mixture.

43. The method of claim 40 wherein said step of adjusting the temperature of said methane or natural gas and/or said O₂ containing gas prior to contacting said catalyst includes maintaining the temperature of the reactant gas mixture at about 600-1,200° C. when contacting said catalyst.

44. The method of claim 40 wherein said step of adjusting the operating pressure of said reactor comprises maintaining said reactant gas mixture at a pressure in excess of 100 kPa (about 1 atmosphere) while contacting said catalyst.

45. The method of claim 44 wherein said pressure is up to about 32,000 kPa (about 320 atmospheres).

46. The method of claim 45 wherein said pressure is between 200-10,000 kPa (about 2-100 atmospheres).

47. The method of claim 40 wherein said step of adjusting the concentrations of methane or natural gas and O₂ in said reactant gas mixture comprises mixing methane or natural gas and an O₂ containing gas to provide a reactant gas mixture having a carbon:oxygen ratio of about 1.25:1 to about 3.3:1.

48. The method of claim 47 wherein said mixing comprises mixing together said methane or natural gas and said O₂-containing gas in a carbon:oxygen ratio of about 1.3:1 to about 2.3:1.

49. The method of claim 48 wherein said mixing comprises mixing said methane or natural gas and said O₂-containing gas at a carbon:oxygen ratio of about 1.5:1 to about 2.3:1.

50. The method of claim 49 wherein said mixing comprises mixing said methane or natural gas and said O₂-containing feedstock at a carbon:oxygen ratio of about 2:1.

51. The method of claim 29 wherein said natural gas comprises at least about 80% methane by volume.

52. The method of claim 29 comprising contacting said reactant gas mixture with a catalyst prepared by a process comprising:
combining in a mixing vessel
at least one decomposable precursor compound of a catalytically active metal or metal oxide,
optionally, at least one decomposable precursor compound of a refractory metal oxide support,
at least one combustible organic compound,
optionally, a liquid mixing agent, such that a mixture is formed;
in an evaporator, evaporating said liquid mixing agent, if present, and/or a portion of said combustible organic compound to produce a catalyst intermediate;
in a furnace, heating said catalyst intermediate to the point of autoignition, and allowing said catalyst intermediate to combust, such that a combustion product is produced;
optionally, calcining said combustion product;
optionally, in a shaping unit, forming said combustion product into a predetermined shape; and
optionally, in an activation unit, heating said combustion residue under activating conditions, to provide an activated catalyst.
